(19) United States
(12) Patent Application Publication
(10) Pub. No.: US 2002/0038185 A1
(43) Pub. Date: Mar. 28, 2002

(54) Method for indicating relationship between cDNA sequence and genome recording medium, sequencer apparatus, and method for designing a primer

(75) Inventors: Kouichi Kimura, Koganei (JP); Tetsuo Nishikawa, Tokyo (JP)

Correspondence Address: Stanley P. Fisher, Reed Smith Hazel & Thomas LLP, Suite 1400, 3110 Fairview Park Drive, Falls Church, VA, 22042-4503, US

(73) Assignee: Hitachi, Ltd.

(21) Appl. No.: 09/933,168

(22) Filed: Aug. 21, 2001

(30) Foreign Application Priority Data
Sep. 25, 2000 (JP) 2000-289728

(51) Int. Cl. G06F 19/00; G01N 33/48

(52) U.S. Cl. 702/20

(57) ABSTRACT

The present invention provides a method for graphically indicating a correspondence between cDNA and genome sequences having an exon-intron structure to be understood easily. From search results of the similarity between a cDNA and a genome, information on the base positions of both edges of similar subsequence pairs (exon), the similarity value thereof, etc. is extracted. From such information, information on subsequence pairs determined unlikely to be significant in view of the similarity value, base length, etc., is eliminated. Furthermore, the conformity of orientation and order between exons is examined, and only an exon covering the cDNA by not less than a qualified ratio and whose correspondence to the cDNA is clear is selected. The selected exon is indicated by a segment on a graph by locating a base position on the genome sequence to an axis 1 and a base position on the cDNA sequence to another axis of the graph, thereby confirming the intron-exon structure visually as a line of segments.

### FIELD OF THE INVENTION

The present invention relates to an analysis of information on a gene sequence, and to a method for deducing and indicating the position and structure of a gene on a genome based on the result of a similarity search between cDNA and genome sequences.

### BACKGROUND OF THE INVENTION

As the method for deducing the position of a gene on a genome and its exon-intron structure, there is a method in which cDNA and genome sequences are subjected to a similarity search, and subsequence sections having similarity are enumerated. Those sections are sorted and enumerated in order of high similarity value. The similarity value is evaluated by a probability that such a similarity appears by chance, and the less the probability is, the higher the evaluation for the similarity value.

This sorting method is useful due to the following reason. A genome of an organism has evolved by deriving and differentiating a copy of a gene. Therefore, in one cDNA sequence, there exist subsequences having various similarity values at a plurality of places on the genome. Among those genome subsequences, the genome subsequence transcribed into mRNA actually used as a template of the cDNA is limited to the one having the highest similarity value. A mismatching portion may result from polymorphism of e.g. SNP, or a sequencing error. Thus, sections having similarity are sorted and enumerated in the order of high similarity value to enumerate subsequences on the genome transcribed into MRNA used as a template of the cDNA in the upper reaches, thereby making it easy to relate cDNA sequences to genome sequences.

With regard to the correspondence between those sequences, a cDNA sequence rarely corresponds to a subsequence on a genome for its entirety as one sequence. Generally, it is separated into several subsequences, and each subsequence corresponds to the subsequence on the genome. Such a correspondence is due to a phenomenon called splicing upon synthesis of mRNA from a genome in eukaryotes including a human. Each subsequence on a genome corresponding to a cDNA is referred to as an exon. Exons are continuously connected to each other on a cDNA, however, they are connected holding subsequences referred to as introns between them on a genome The positional relationship between an exon on a cDNA and that on a genome is as described in either (1) or (2) below.

(1) A sequence of each exon on a CDNA and that on a genome are almost identical (this will be mentioned as having the same orientation hereinafter), and they are lined up in the same order.

(2) A sequence of each exon on a cDNA are nearly complementary to that on a genome (this will be mentioned as having the opposite orientation hereinafter), and they are lined up in the opposite order to each other.

The aspect of a correspondence between cDNA and genome sequences having such an exon-intron structure cannot be comprehended only by enumerating sections having similarity, therefore the positions of those sections to each other must be examined. For that purpose, a two-dimensional plot is useful in which a base position on a genome sequence and that on a cDNA sequence are located to both axes. Examples of the simplest plotting methods include a dot matrix method in which dots are plotted on coordinates (x, y) in two-dimension in the case where the base position “x” on a genome sequence and the base position “y” on a CDNA sequence are identical (p. 105, Sequence Analysis Primer, M. Gribskov and J. Devereux, Oxford University Press, 1992). This method enables a detailed comparison locally. Alternatively, examples of methods for comprehending the relationship of the correspondence in a broader perspective include a method comprising: locating windows of a qualified base length in genome and cDNA sequences; locating the position of the window in the genome sequence to x-axis, and that in the cDNA sequence to y-axis in the case where the similarity of base sequences in those windows is not less than a qualified ratio; thereby plotting line 2segments corresponding to those windows on a two-dimensional plane (p. 108, Sequence Analysis Primer, M. Gribskov and J. Devereux, Oxford University Press, 1992). By this method, comparison is not performed for an individual base, rather, an average comparison is performed for several to dozens of bases, enabling the comparison between longer sequences and allowing elimination of non-significant short identical portions accidentally appeared.

The corresponding relationship between cDNA and genome sequences having an exon-intron structure is shown graphically to be easily understood. There are regions on a genome where a number of genes exist, to which a number of cDNAs correspond (also mentioned as “attach”). Those positional relationships will be easily understood visually when graphically indicated.

In the exon-intron structure of a gene, an intron sequence may be extremely longer than an exon sequence. The length of a cDNA sequence is approximately from several hundreds- to tens of thousands-base length, however the corresponding gene region on a genome may extend to an order of a million base length. Thus, in the case where lengths of cDNA and genome sequences to be corresponded differ by as many as three orders, the conventional method in which the same sized windows are moved for examination in cDNA and genome sequences is inefficient.

In the case where the position of a sequence similar to cDNA is indicated throughout a wide region on a genome, a number of similar sequences not being involved in a true corresponding relationship will appear, prohibiting the true relationship to be selected out of the two dimensional indication. Examples of such sequences include a similar short sequence, a sequence having low similarity value, and a similar sequence having mismatching orientation or order. Accordingly, elimination of those unnecessary similar sequences will be required.

### SUMMARY OF THE INVENTION

In the present invention, relative to given cDNA and genome subsequences, the corresponding relationship between them having an exon-intron structure is indicated by a method comprising the following processing steps of:

(1) arranging given cDNA sequences to build a database for searching; and repeatedly performing similarity searches relative to the database of CDNA sequences using each given genome subsequence as a query sequence.

(2) enumerating pairs of cDNA and genome subsequences similar to each other to calculate the following as their characteristics:

i base length of a subsequence

ii similarity value

iii orientation and order of each subsequence on genome or CDNA sequences

iv coverage ratio of a cDNA subsequence with respect to covering the entire cDNA sequence in cooperation with the cDNA subsequence of other pairs

(3) eliminating a subsequence pair from a set of subsequence pairs having similarity enumerated in the above item, the subsequence pair not satisfying qualified loose requirements given regarding the above-described characteristics. This aims at eliminating a subsequence pair with low possibility of reflecting meaningful similarity, thereby compressing a processing volume. Specifically, the subsequence pair to be eliminated is a subsequence pair under a qualified length or similarity value, subsequence pairs incapable of having matching orientation and order to each other on a genome, or a subsequence pair having no possibility of covering a CDNA sequence by not less than a qualified ratio in cooperation with other subsequence pairs.

(4) filtering a set of pairs to be indicated from sets of subsequence pairs with similarity enumerated in the above item by a more strict requirement relative to the above-mentioned characteristics. This aims at accurately selecting pairs having high possibility of reflecting meaningful similarity. For that purpose, for example, using a graphical indication, parameters giving a threshold of a requirement for filtering the set are adjusted by an interactive instruction from a user. Alternatively, a set of subsequences which appear in the matching orientation and order, the subsequences being capable of covering a cDNA sequence by not less than the qualified ratio in cooperation with other pairs, is automatically selected according to a program, and the results are graphically indicated.

(5) two-dimensionally indicating the positional relationship between the pair of selected cDNA and genome subsequences. A base position on the genome sequence is located to the axis 1 of a graph and a base position on the cDNA sequence is located to another axis, indicating each subsequence pair by a piece of line segment. This segment indicates the position of a subsequence when projected to each axis and the orientational correspondence of the cDNA and genome.

Therefore, the method of the present invention for indicating the correspondence between the cDNA and genome sequences is characterized by:

locating the base position on the genome on an axis 1 of a graph, and the base position on the cDNA sequence on another axis; and

indicating a portion having similarity to said cDNA sequence of not less than the qualified ratio by a segment on a graph, in the subsequence having not less than the qualified base length among said genome sequences.

It is preferable to locate a plurality of cDNAs on a vertical axis and indicate the corresponding relationship with said cDNAs using a different color for each cDNA.

The present invention is also a recording medium readable by a computer in which a program for executing, by a computer, a method for indicating a correspondence with cDNA and genome sequences is recorded, the method comprising the following steps of: inputting genome and cDNA sequences; searching a portion having similarity to said cDNA sequence of not less than the qualified ratio, in a subsequence having not less than the qualified base length among said genome sequences; locating said genome and cDNA sequences on vertical and horizontal axes or horizontal and vertical axes of a graph, respectively, and indicating the portion searched in said searching step by a line segment on a graph.

Furthermore, the method for indicating the correspondence between cDNA and genome sequences preferably comprises a step of inputting the qualified base length and the qualified similarity ratio.

Still further, the sequencer apparatus of the present invention comprises: a means of accessing to a genomic database connected with a network or to an internal database to input a genome sequence, and inputting cDNA sequence obtained by sequencing thereto; a means of searching a portion having not less than the qualified similarity ratio to said cDNA sequence, in the subsequence having not less than the qualified base length among said genome sequences; a means of indicating the portion searched by said searching means by a line segment on a graph in which the genome and cDNA sequences are located on vertical and horizontal axes or horizontal and vertical axes, respectively, thereby indicating an exon-intron structure of a gene on the genome sequence corresponding to the cDNA sequence.

A method for designing primers of the present invention comprising the following steps of: designing primer pairs in different exon regions holding an intron sequence between them and performing PCR by using the primers with genome and cDNA libraries, respectively; inputting genome and cDNA sequences amplified by said PCR step; searching the portion having not less than the qualified similarity ratio to said cDNA sequence, in the subsequence having not less than the qualified base length among said genome sequences; indicating the portion searched by said searching step by a line segment on a graph in which said genome and cDNA sequences are located on vertical and horizontal axes or horizontal and vertical axes, respectively, thereby indicating that different polynucleotides have been amplified due to the presence of an intron sequence and confirming that the amplified genome sequence comprises the intron sequence.

### BRIEF DESCRIPTION OF THE DRAWINGS

FIG. 1 shows the processing flow in an embodiment of the present invention.

FIG. 2 shows a data structure of information where similar subsequence pairs (exon) have been gathered.

FIG. 3 shows a flow chart for explaining the performance of the primary selecting process of pairs of similar subsequences (exon).

FIG. 4 is an illustration briefly indicating an image depicted on a monitoring display.

FIG. 5 shows a flow chart for explaining the performance of the secondary selecting process of pairs of similar subsequences (exon).

FIG. 6 is a drawing explaining a principle of a method for designing primers in the second embodiment of the present invention.

### DESCRIPTION OF THE PREFERRED EMBODIMENTS

The embodiment of the present invention will be described in detail as follows using the drawings.

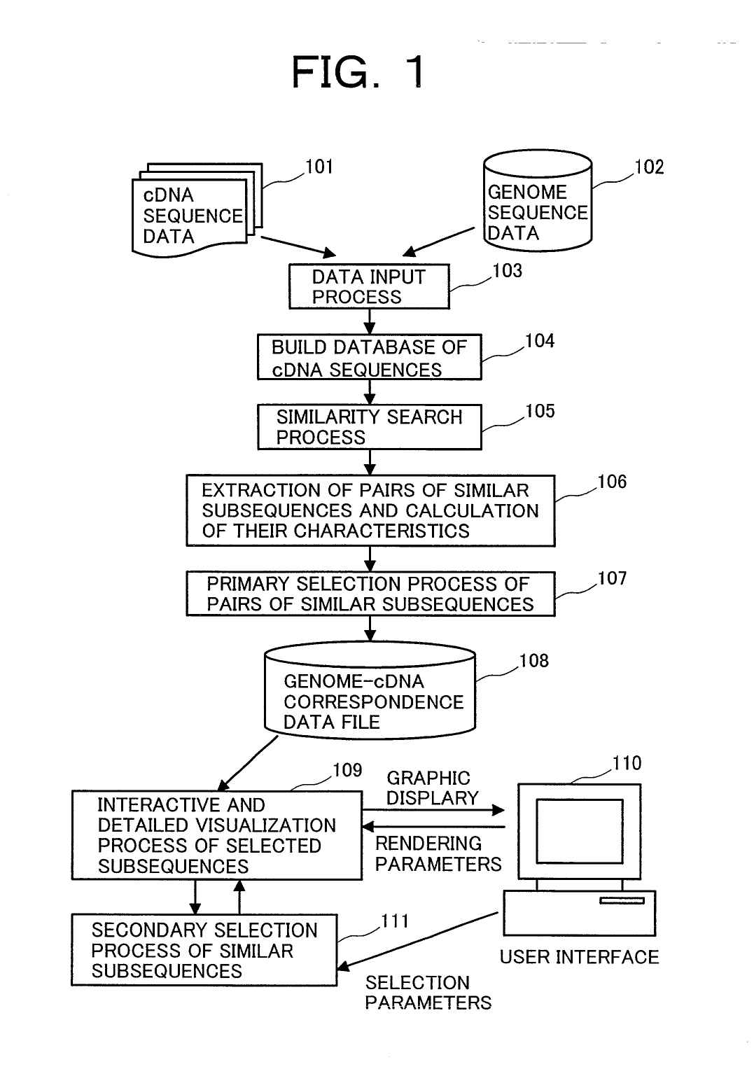

FIG. 1 shows the processing flow in an embodiment of the present invention, in which a given cDNA sequence is attached to a genome sequence in a database, thereby aiming at visualizing an exon-intron structure of the gene corresponding to the cDNA.

In FIG. 1, 101 shows a cDNA sequence data directed to an analysis and 102 shows a database in which a genome sequence to be compared to a cDNA sequence is stored. 103 shows an input process for reading a database of cDNA and genome sequences. 104 shows a process for creating a database of the entered cDNA sequence data for the preparation of the following similarity search by a program “formatdb” using a known method (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402). 105 is a process for repeatedly performing similarity searches to the cDNA database using each genome segment sequence in a genome database as a query sequence. Each similarity search is performed by BLAST using a known algorithm (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402). 106 is a process comprising: reading all text data in which similarity search results obtained from each genome subsequence are described; extracting and enumerating similar subsequences appearing therein; and calculating various volumes characterizing each subsequence. 107 is the primary selecting process of pairs of similar subsequences where the subsequence satisfying the qualified loose requirements is selected from enumerated similar subsequences based on those various characteristics. This aims at eliminating subsequences having a low possibility of reflecting a significant similarity and compressing the volume to be processed. The selection results are stored in a file 108. Since the calculation process mentioned so far is time-consuming and these calculations are performed only once independently from a subsequent interactive process with a user, the results are thus stored in a file. 109 is a process comprising: reading from the file 108 of a positional relationship to each other of subsequences selected from the similar subsequences on cDNA and genome; and generating a two-dimensionally indicated graphical data, allowing the data to be easily understood by users. 110 is a user interface apparatus equipped with a monitoring display, keyboard and mouse, which indicates the graphic data generated by 109 and also accepts a rendering parameter from a user, transmitting it to 109 to effect recalculation of the graphic data, thus 109 and 110 cooperate to allow an interactive indication. Furthermore, 111 is the secondary selecting process of similar subsequences, which further filters subsequences by a more strict requirement. This aims at more accurately selecting subsequences possibly reflecting a significant similarity. 110 accepts parameters necessary for that purpose from users and transmits them to 111. Data of similar subsequences further filtered by 111 are transmitted to 109 where the graphic data are recalculated. This is retransmitted to 110 and indicated to users. By 109, 110 and 111, a method for selecting subsequences can be altered interactively, thereby making it possible to select a set of subsequences accurately representing the corresponding relationship between a genome and a cDNA.

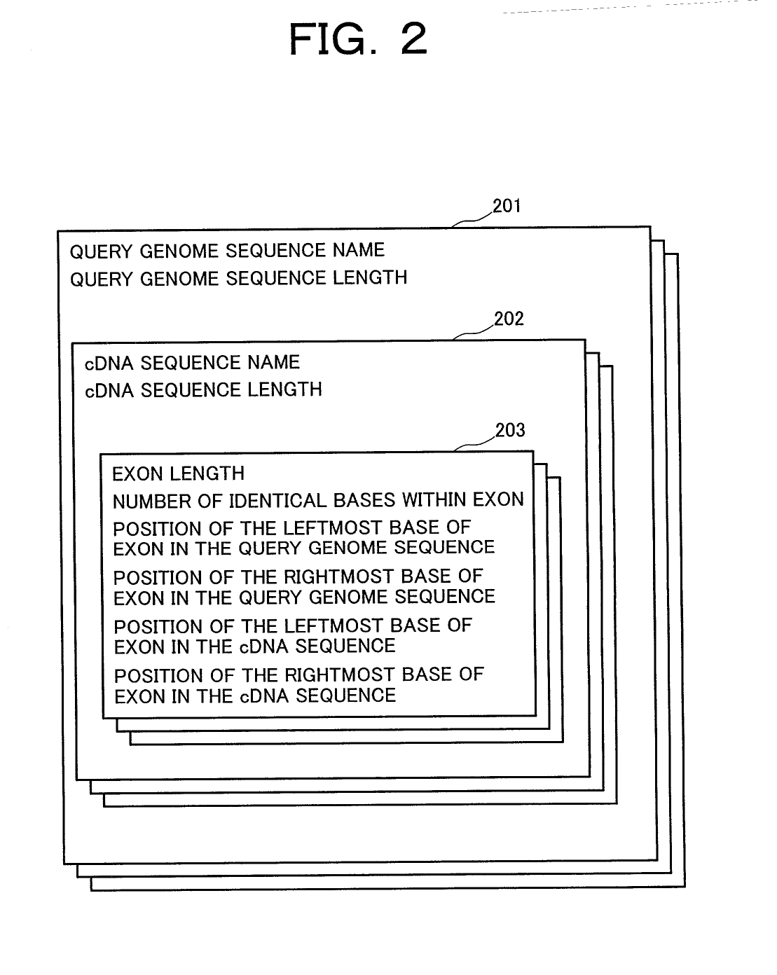

FIG. 2 shows a data structure obtained in 106 by extracting similar subsequence pairs between subsequences of genome subsequences and cDNA subsequences. All of the information appearing therein can be obtained from the similarity search results by BLAST program in 105. 201 is a data corresponding to a single genome subsequence, and the whole data has repetitions of this structure. 201 at least comprises a repetitive structure of information 202 related to a name for identifying a sequence of a genome fragment and the length thereof, and cDNA having a subsequence similar to the sequence of the genome fragment. 202 at least comprises a repetitive structure of information 203 related to a name for identifying a cDNA and length of the sequence thereof, and subsequence similar to the genome. Hereinafter, for simplifying an explanation, the subsequences in a genome and cDNA which are similar to each other will be referred to as “exon.” This term will correspond not only to a biological exon but also to a pair of similar subsequences having contingently appeared. 203 is information on exons, at least comprising information on lenght, number of identical bases between genome and cDNA, and position in a genome subsequence and a cDNA sequence.

The data structure shown in FIG. 2 is a basic structure of information processed in and after 106 in FIG. 1, and information stored in file 108 also has this data structure. This is the structure in which a part of information judged as having low usability in 107 is eliminated from information obtained in 106. 109 reads information having the data structure shown in FIG. 2 and indicates it graphically; 111 reads information having the data structure shown in FIG. 2 and selects the exon judged as having high usability therefrom, then returns again the information of the data structure shown in FIG. 2 to 109.

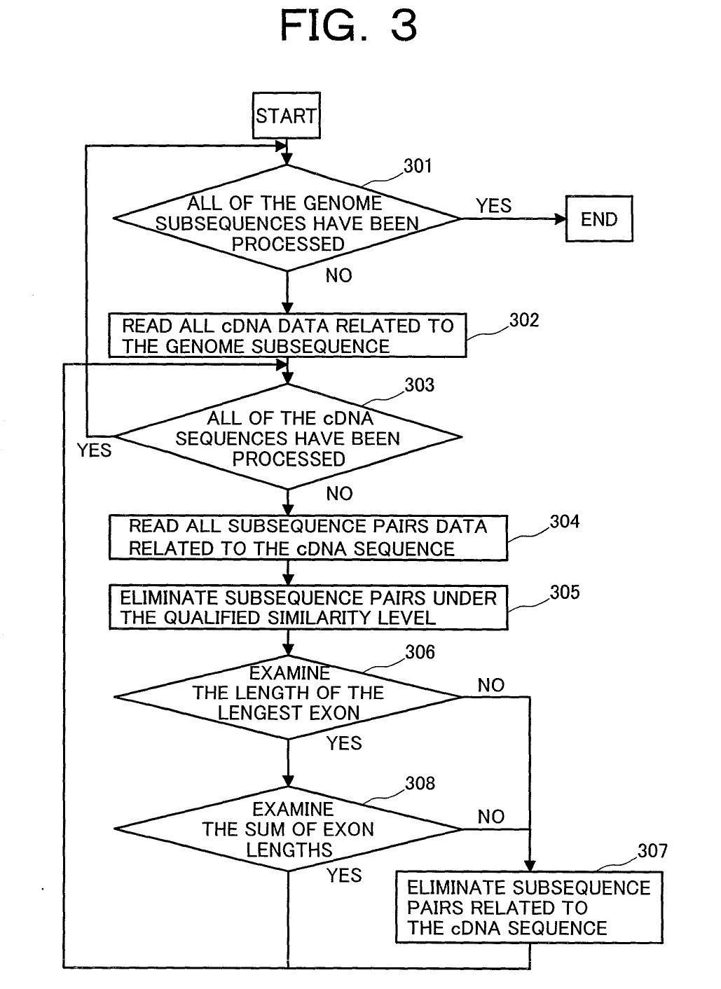

FIG. 3 is a flow chart explaining the performance of the primary selection process of pairs of similar subsequences (exon) of 107. By performing the repetitive process including an end judgment by 301, the following process is performed to all of the genome subsequences. 302 reads information shown in 201 related to genome subsequences under process. A plurality of information on CDNA shown in 202 is included therein. By performing the repetitive process including an end judgment by 303, the following process is performed to all cDNA. 304 reads information shown in 202 related to cDNA sequences under process. A plurality of information on an exon shown in 203 is included therein. 305 calculates the similarity value of each exon by the equation:

(Similarity value)=(the number of identical bases in exon)/(exon base length);

and in the case where the resultant value is under the qualified similarity value, the corresponding exon is eliminated from the enumerated exons in 203. If 80% is set as the qualified similarity value, for example, most of genome fragment subsequences, excepting exons, contained in the gene used as a template of cDNA presently under process (or the closely related gene) are considered to be eliminated. Subsequently, 306 calculates the maximum length of a remaining exon and judges whether it is not less than the qualified value. In most cases, there is at least one exon having approximately 100 base-length among exons in a gene. Therefore, for example, when there is no exon having approximately 50 base-length, it is considered that there is a high possibility that a portion of a repetitive sequence unevenly distributed abundantly in a genome has been taken. Accordingly, all of the exon information and the cDNA information thereof are eliminated by 307. If it is not the case, the total exon length is calculated to find the ratio to the full length of the cDNA sequence, thereby judging whether the value is no less than the qualified value by 308 . When the value of the ratio is below 30%, for example, those exons can cover only a slight portion of the cDNA sequence, meaning that the relationship between the cDNA and genome therein is tenuous. Accordingly, all exon information and the cDNA information thereof are eliminated.

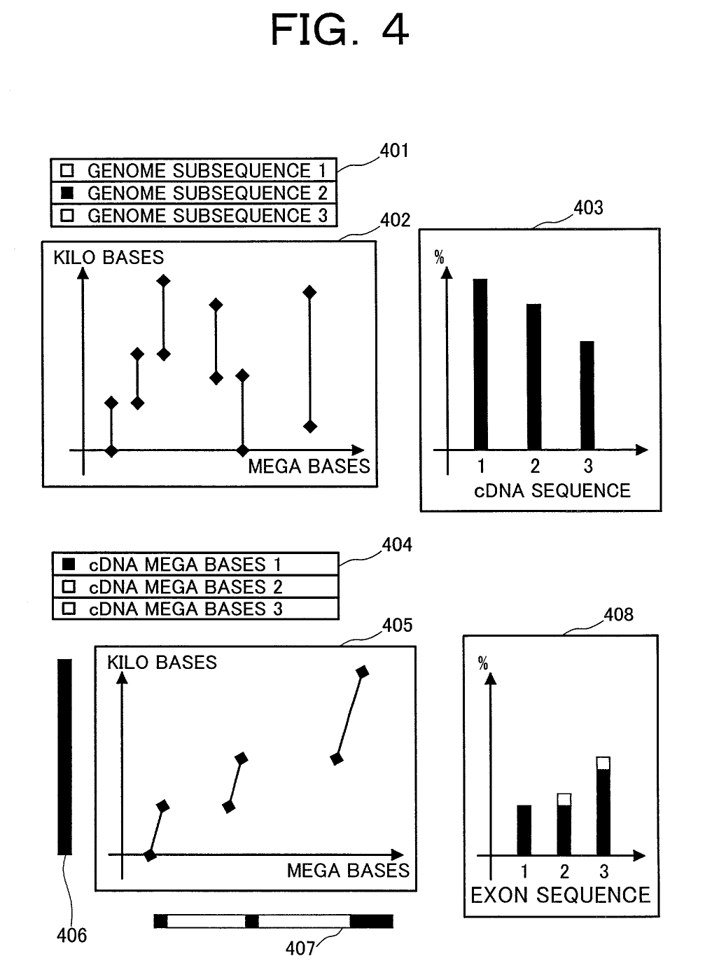

FIG. 4 is an illustration briefly indicating an image generated by the indicating process of 109 and rendered on the monitoring display of 110. 401 is a list of processed genome subsequences, and shows that one of the items (“genome subsequence 2” in the drawing) is selected and the result of an analysis thereof is indicated on the monitoring display. 402 shows with a segment an exon which indicates a pair of similar subsequences between a genome and a cDNA, by locating the base position on the genome subsequence to the horizontal axis with a rough coordinate system (mega base unit in the drawing), and the base position on the cDNA sequence to the vertical axis with a detailed coordinate system (kilo base unit in the drawing). These exon-indicating segments are indicated using a different color for each cDNA on the actual monitor display. 403 shows what percentage of the entire cDNA sequence the united exons cover, relative to each cDNA. This indicates how closely related the cDNA is to the genome subsequence presently under process. 404 is a list of cDNA sequences, and shows that one of the items (“cDNA sequence 1” in the drawing) is selected and the result of an analysis thereof is indicated on the monitor display. Relative to the cDNA selected by 404, 405 enlarges a partial plot of 402 containing the cDNA. 406 shows the plot of a segment indicating the exons of 405 being projected on the vertical axis, hereby confirming to what extent the united exons cover the entire cDNA. 407 shows the plot of the segment indicating the exons of 405 being projected on the horizontal axis. The portion between projected exons indicates an intron. 408 indicates the base length and the number of identical bases therein (between genome and cDNA) relative to each exon, thereby confirming how high the similarity value is between the genome and cDNA in each exon.

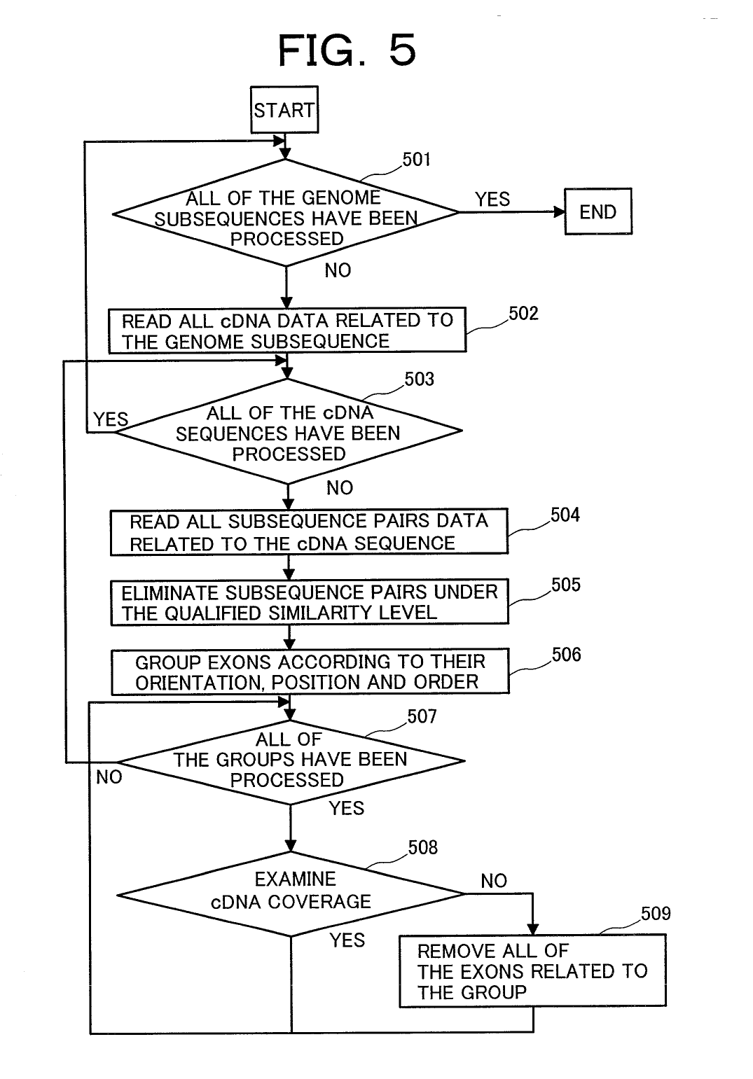

FIG. 5 is a flow chart explaining the performance of the secondary selection process of pairs of similar subsequences (exon) of 111. By performing the repetitive process including an end judgment by 501 , the following process is performed to all genome subsequences. 502 reads information shown in 201 related to the genome subsequence under process. A plurality of information on the cDNA shown in 202 is included therein. By performing the repetitive process including an end judgement of 503, the following process is performed to all of these cDNA. 504 reads information shown in 202 related to the cDNA sequence under process. A plurality of information on an exon shown in 203 is included therein. 505 calculates the similarity value of each exon by the equation:

(Similarity value)=(the number of identical bases in an exon)/(exon base length);

and in the case where the resultant value is under the desired similarity value, the corresponding exon is eliminated from the enumerated exons in 203. The desired similarity value is transmitted to the program by a user interface 111. For example, if a similarity value of 98% is required here, it is considered that only an exon contained in a gene used as a template of the cDNA presently under process (or a gene closely related thereto) will be selected, allowing that the difference of the order of 2% is due to a SNP polymorphism or sequencing error. Subsequently, 506 divides the set of the remaining exons into groups in which the orientation and order are matching. In each group, the set of exons belonging thereto satisfy any of the following conditions:

(1) each exon sequence on a cDNA and each one on a genome are almost identical (referred to as having the same orientation, or forward orientation), and they are lined up in the same order.

(2) each exon sequence on a cDNA and each one on a genome are in an almost complementary relationship to each other (referred to as having the opposite orientation, or reverse orientation); and they are lined up in the opposite order. A procedure to perform such grouping is described later. By performing a repetitive process including an end judgment by 507, the following process is performed relative to each group of exons. 508 calculates the ratio of the entire cDNA covered by the united exons belonging to the same group to examine whether it is no less than the qualified ratio (e.g. 95%), and determines whether the interval between adjacent exons is less than the qualified base length (e.g. 10 bases) when exons belonging to the same group are lined up in ascending order. When any nonobservance is confirmed, in 509 all exons belonging to that group are eliminated from 203.

The grouping of the entire exons belonging to one cDNA as in 506 above is performed according to the following procedures. First, the entire exons belonging to one cDNA are divided into two groups depending on the orientation (forward/reverse.) Then, the exons in the forward orientation are sorted in ascending order depending on their position on a genome subsequence, and the exons in the reverse orientation are sorted in descending order depending on their position on a genome subsequence. Exons in each orientation are observed in the order of sorting, and:

(1) the first exon belongs to a new group;

(2) if the following equation holds for the present exon q relative to the proximate exon p, (the position of the q rightmost base on the cDNA sequence) >(the position of the p rightmost base on the cDNA sequence)−(the number of allowable overlap bases), q belongs to the same group as p; and if this is not the case, q belongs to a new group. The number of allowable overlap bases may be of the order of 5 bases, for example.

### Example 2

Using the indication of correspondence between cDNA and genome sequences as in the above example, the second embodiment of the present invention for designing primers will be explained using the drawings.

Generally, when a cDNA library is created, other genomic fragments other than cDNAs may be mixed in as a polynucleotide included therein. Accordingly, when a part of a cDNA sequence is amplified by PCR, it is useful to confirm that it is an actual part of the cDNA sequence, not the sequence of other genome fragments.

Use of the above example in the designing of primers will enable such confirmation.

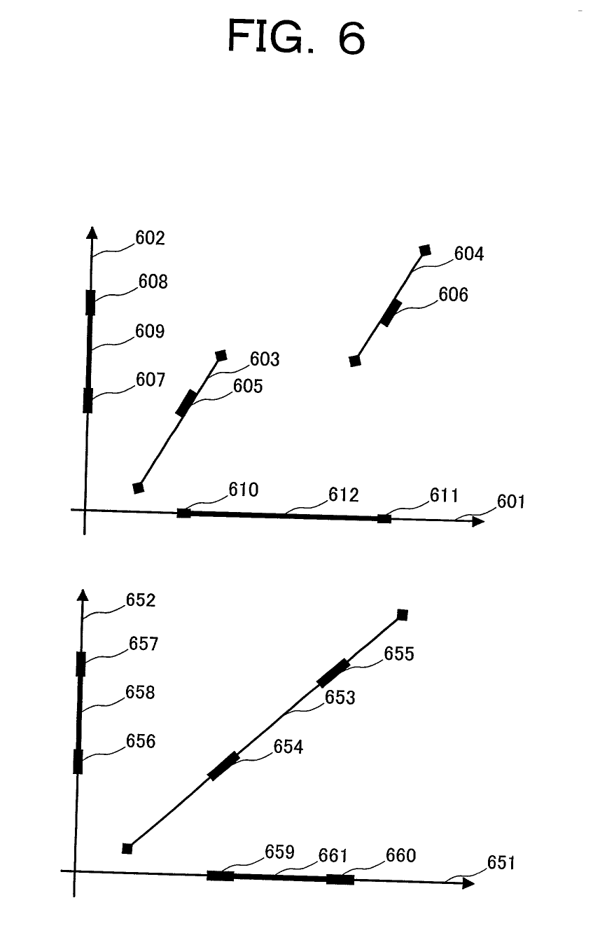

FIG. 6 is a drawing of the principle, explaining a method for designing such primers. 601 is an axis indicating the base position on a genome; 602 is an axis indicating the base position on cDNA; 603 and 604 indicate different exons belonging to one cDNA. A primer sequence is selected from base sequences of 603 and 604 according to a known method (Tahira, Hayashi, PCR, PCR-SSCP, new handbook of gene-engineering, Muramatsu and Yamamoto eds., 75, Yodosha, 1999.) If an oligonucleotide of this primer sequence is synthesized and PCR is performed for a cDNA library, these primers will bind to CDNA(s) at positions of 607 and 608, amplifying a polynucleotide having a cDNA subsequence between them shown as 609. On the other hand, if PCR is performed for a genome library using the same primers, these primers will bind to the genome at positions of 610 and 611, amplifying a polynucleotide having a genome subsequence between them shown as 612. This polynucleotide comprises an intron sequence. Thus, polynucleotides amplified by these two PCRs are different in their lengths.

On the contrary, when primers are (undesirably) designed from a genome fragment mixed in with the cDNA library, polynucleotides amplified by two types of PCR as in the above will be identical. 651 is an axis showing a base position on a genome, 652 is an axis showing a base position on cDNA, and 653 shows an exon. A primer sequence is selected from base sequences of 653. If the oligonucleotide of this primer sequence is synthesized and PCR is performed for a cDNA library, these primers will bind to the genome fragment contained in the cDNA library at positions of 656 and 657, amplifying a polynucleotide having a subsequence between them shown as 658. On the other hand, if PCR is performed for a genome library using the same primers, these primers will bind to the genome at positions of 659 and 660, amplifying a polynucleotide having a subsequence between them shown as 661. Thus, polynucleotides amplified by these two types of PCR are identical.

As mentioned in the above, by examining the difference of polynucleotides amplified by PCR for cDNA and genome libraries using the same primers, it can be confirmed that a part of the cDNA, and not a genome fragment mixed in with a cDNA, was amplified.

The corresponding relationship between cDNA and genome sequences having an exon-intron structure is graphically indicated as segments of matching orientation and order (corresponding to an exon) so as to be comprehended easily. For pairs of similar subsequences that are candidates for an exon, items such as the base positions of both edges and the similarity value thereof are calculated in advance to allow a broad range of rendering at high speed to interactively select and render pairs of similar subsequences more likely to be an exon from among the candidates. Since sequences such as a short similar sequence, a similar sequence having a low similarity value, or a similar sequence of mismatching orientation and order are automatically eliminated for indication, only significant corresponding relationships between cDNA and genome sequences are depicted.

What is claimed is:

1. A method for indicating a correspondence between cDNA and genome sequences wherein the method comprises: locating a base position on a genome sequence to an axis 1 of a graph, and a base position on a cDNA sequence to another axis; and indicating by a segment on a graph a portion having a similarity of not less than a qualified ratio to the cDNA sequence, in a subsequence in the genome sequence having a base length of not less than a qualified base length.

2. The method for indicating the correspondence between cDNA and genome sequences of claim 1, wherein a plurality of cDNAs are located to a vertical axis and the corresponding relationships to the cDNAs are indicated using a different color for each cDNA.

3. A computer readable recording medium wherein a program is recorded which makes a computer execute the method for indicating the correspondence between cDNA and genome sequences, the method comprising the following steps of: inputting genome and cDNA sequences; searching a portion having a similarity of not less than a qualified ratio to the cDNA sequence, in a subsequence in the genome sequence having a base length of not less than a qualified base length; indicating the portion searched in the search step by a segment on a graph by locating the genome and cDNA sequences to vertical and horizontal axes or horizontal and vertical axes of the graph, respectively.

4. The recording medium of claim 3 wherein a program is recorded which makes a computer execute the method for indicating the correspondence between cDNA and genome sequences, the method further comprising a step of inputting the qualified base length and the qualified ratio of similarity.

5. A sequencer apparatus comprising means of: inputting a genome sequence by an access to a genome database connected to a network or to an internal database, and inputting a cDNA sequence obtained by sequencing; searching a portion having a similarity of not less than a qualified ratio to the cDNA sequence, in a subsequence in the genome sequence having a base length of not less than a qualified base length; indicating by a segment on a graph the portion searched in the above search step by locating the genome and cDNA sequences to vertical and horizontal axes or horizontal and vertical axes of the graph, respectively, thereby indicating an exon-intron structure of a gene on the genome sequence corresponding to the cDNA sequence.

6. A method for designing primers comprising the following steps of: designing primer pairs in different exon regions holding an intron sequence between them, performing PCR by using the primers with genome and cDNA libraries, respectively; inputting genome and cDNA sequences amplified by the PCR step; searching a portion having the similarity of not less than a qualified ratio to the cDNA sequence, in a subsequence in the genome sequence having a base length of not less than a qualified base length; indicating the portion searched in the above search step by a segment on a graph by locating the genome and cDNA sequences to vertical and horizontal axes or horizontal and vertical axes of the graph, respectively, to show that a different polynucleotide has been amplified due to the presence of an intron sequence, thereby confirming the amplified genome sequence contains the intron sequence.
